(19) United States
(12) Patent Application Publication
(10) Pub. No.: US 2005/0177199 A1
(43) Pub. Date: Aug. 11, 2005

(54) PSA cable and connector for quadripolar lead terminal

(75) Inventors: David Hansen, Oakdale, MN (US); Christine Wood, Orono, MN (US); David Yingling, Stillwater, MN (US); Michael A. Knipfer, Maple Grove, MN (US); Gregory L. Sundberg, Stillwater, MN (US)

Correspondence Address: Merchant & Gould P.C., P.O. Box 2903, Minneapolis, MN, 55402-0903, US

(73) Assignee: Cardiac Pacemakers, Inc.

(21) Appl. No.: 10/776,661

(22) Filed: Feb. 9, 2004

(51) Int. Cl. A61N 1/375

(52) U.S. Cl. 607/37; 607/115

(57) ABSTRACT

An adapter for an implantable cardiac lead, the adapter having a housing with an interior axial opening and an outer surface. The interior axial opening is configured to receive a mating terminal end of the implantable cardiac lead. A plurality of inner contacts are positioned within the interior axial opening and a plurality of outer contacts are positioned on the outer surface. Each of the inner contacts is electrically connected to an outer contact. The inner contacts are configured to electrically link with contacts on the mating terminal end of the implantable cardiac lead when the mating terminal end of the implantable cardiac lead is positioned within the interior opening. The outer surface is configured for connection of the outer contacts to a testing cable, the testing cable providing electrical connectivity with a device for testing the electrical continuity of the cardiac lead. An assembly including an adapter may be used to connect implanted cardiac leads with a testing device.

### TECHNICAL FIELD

The present invention relates generally to devices for testing implanted electrical leads. More specifically, the present invention relates to devices for testing multi-conductor electrical leads implanted in the heart of a patient.

### BACKGROUND

A common treatment for heart rhythm disorders and other forms of heart disease is the placement of a pulse generating device such as a pacemaker in the patient's body. Pacemakers and other pulse generating devices rely on electrical leads to receive rhythm information from the heart and to deliver therapeutic rhythm altering electrical signals to the heart. Such leads are implanted within the heart during a surgical procedure and are tested for proper positioning and function as part of the implantation process prior to being connected to the pulse generating device.

These implanted leads include a conductor end in contact with the heart and a terminal end connected with the pulse generator. The proximal terminal end typically includes one or more exposed contacts electrically connected with the distal conductor end. During the testing process, a Pacing System Analyzer (PSA) may be connected with certain contacts of the terminal end to test for proper function and connection of the conductor end to the heart. Prior art standards for implanted leads, such as IS-1 Standards, include two conductors with exposed contacts at the terminal end and the PSA is electrically with these contacts to test the lead.

New potentially forthcoming quadripolar lead standards for implanted leads include up to four conductors within the lead and up to four contacts at the terminal end of the lead. The terminal end of a quadripolar lead is not significantly different in size from the terminal end of an IS-1 lead. Thus, quadripolar leads have twice as any many contacts in the same size terminal end. Existing methods of connecting the PSA to the contacts and conductors of IS-1 leads are not entirely satisfactory for connecting the PSA to the appropriate contacts and conductors of a quadripolar lead due to tighter axial spacing of the contacts of a quadripolar lead.

For example, currently, it is known to attach alligator or similar spring loaded clips about the terminal end of an IS-1 lead in direct contact with the contacts of the terminal end. The same clips may not be satisfactory for use with a quadripolar lead as the contacts of the terminal end of the quadripolar lead are too densely situated. The clips might come into contact with each other, which would provide inaccurate PSA testing results. Also, placement of clips directly onto the terminal end may cause undesirable deformation of the surface of the terminal end.

Improvements to the connecting of a PSA to contacts and conductors of a terminal end of an implanted electrical lead are desirable.

### SUMMARY

The present invention relates generally to the connection of testing devices to implanted cardiac leads. More specifically, the present invention relates to an adapter for an implantable cardiac lead with a housing having an interior axial opening and an outer surface. The interior axial opening is configured to receive a mating terminal end of the implantable cardiac lead. A plurality of inner contacts are positioned within the interior axial opening and a plurality of outer contacts are positioned on the outer surface. Each of the inner contacts is electrically connected to an outer contact. The inner contacts are configured to electrically link with contacts on the mating terminal end of the implantable cardiac lead when the mating terminal end of the implantable cardiac lead is positioned within the interior opening. The outer surface is configured for physical and electrical connection of the outer contacts to a testing cable, the testing cable providing communication with a device for testing electrical aspects of the cardiac lead.

The present invention further relates to an assembly for testing an implanted cardiac lead. The cardiac lead includes a distal end in electrical communication with a patient's heart and a proximal end including a terminal end adapted for connection to a pulse generating device, where the terminal end includes a plurality of contacts. The assembly also includes a testing cable adapted to communicate with a device for testing the electrical continuity of the cardiac lead. The testing cable includes a distal end with a plurality of spring clips for linking to the terminal end of the implanted cardiac lead and a proximal end for connection to the testing device. The assembly also includes an adapter positioned about the terminal end of the implanted cardiac lead and providing an electrical connection between the plurality of contacts of the terminal end and the plurality of spring clips mounted at the distal end of the testing cable.

The present invention further relates to a method of connecting an implanted cardiac lead to a testing device. The method includes providing the implanted cardiac lead having a distal end positioned within a body of a patient and a terminal end including a plurality of contacts. A testing cable with a proximal end adapted for connection with the testing device and a distal end including a plurality of spring clips is also provided. Further provided is an adapter including a housing defining an axial opening with a first end and a second end, a plurality of inner contacts within the opening, and a plurality of outer contacts electrically connected with the inner contacts. The terminal end of the cardiac lead is placed within the axial opening of the adapter so that contacts of the terminal end are electrically linked with the inner contacts. The spring clips of the distal end of the testing cable are placed in electrical contact with the outer contacts of the adapter.

### BRIEF DESCRIPTION OF THE DRAWINGS

The accompanying drawings, which are incorporated in and constitute a part of the description, illustrate several aspects of the invention and together with the detailed description, serve to explain the principles of the invention. A brief description of the drawings is as follows:

FIG. 1 is a schematic view of a patient with an implanted pacemaker connected to the patient's heart by a cardiac lead.

FIG. 2 is a side view of a terminal end of a first prior art cardiac lead with a pair of conductors, consistent with an IS-1 Standard.

FIG. 3 is a side view of a terminal end of a first cardiac lead according to the forthcoming quadripolar standard with four conductors.

FIG. 4 is a side view of a terminal end of a second cardiac lead according to the forthcoming quadripolar standard with four conductors.

FIG. 5 is a side view of the terminal end of the cardiac lead of FIG. 2, with alligator spring clips of a PSA testing cable connected to each of the two conductors.

FIG. 6 is a side view of the cardiac lead of FIG. 4, with the alligator spring clips of the PSA testing cable of FIG. 6 connected to two of the conductors.

FIG. 7 is a perspective view of a first embodiment of a testing cable connection adapter for use with the terminal end of the cardiac lead and the alligator spring clips of the PSA testing cable of FIG. 6, with the terminal end positioned for insertion within a central axial opening of the adapter.

FIG. 8 is a side view of the adapter of FIG. 7, with the terminal end of the cardiac lead positioned within the central axial opening.

FIG. 9 is a side of an alternative configuration of the adapter of FIG. 7.

FIG. 10 is a perspective view of a second embodiment of a testing cable connection adapter for use with the terminal end of the cardiac lead and the alligator spring clips of the PSA testing cable of FIG. 5, with the terminal end positioned for insertion within a central axial opening of the adapter.

FIG. 11 is a perspective cross-sectional view of the adapter of FIG. 10.

FIG. 12 is a side view of the adapter of FIG. 10.

FIG. 12A is a side view of the adapter of FIG. 10 connecting the cardiac lead of FIG. 1 to a device for testing the lead.

FIG. 13 is a perspective view of a third embodiment of a testing cable connection adapter for use with the terminal end of the cardiac lead and the alligator spring clips of the PSA testing cable of FIG. 6, with the terminal end positioned for placement with the adapter so that the adapter may be closed about the terminal end.

FIG. 14 is a top view of the adapter of FIG. 13.

FIG. 15 is an end view of the adapter of FIG. 13, with the adapter closed to form a central axial opening.

FIG. 16 is an alternative configuration of the adapter of FIG. 11.

FIG. 17 is a perspective view of a testing cable assembly including a distal end for use with the terminal end of FIG. 5.

FIG. 18 is an enlarged view of the distal end of the testing cable assembly of FIG. 17.

FIG. 19 is a perspective cross-sectional view of the distal end of the testing cable assembly of FIG. 18.

FIG. 19A is a side view of the testing cable assembly of FIG. 17 connecting the cardiac lead of FIG. 1 to a device for testing the lead.

FIG. 20 is a perspective view of a first alternative embodiment of a testing cable assembly including a distal end for use with the terminal end of FIG. 6.

FIG. 21 is an enlarged perspective view of the distal end of the testing cable assembly of FIG. 20.

FIG. 22 is a perspective view of an adapter sleeve according to the present invention mounted about a terminal end of a quadripolar lead, with a spring contact within the sleeve shown in dashed lines.

FIG. 23 is a perspective view of an adapter clip according to the present invention for use with the terminal end of the quadripolar lead.

FIG. 24 is a side view of the adapter clip of FIG. 23.

FIG. 25 is an end view of the adapter clip of FIG. 23, mounted about the terminal end of the quadripolar lead of FIG. 2.

FIG. 26 is a first contact for use in the clip of FIG. 23.

FIG. 27 is a second contact for use in the clip of FIG. 23.

### DETAILED DESCRIPTION

Reference will now be made in detail to exemplary aspects of the present invention which are illustrated in the accompanying drawings. Wherever possible, the same reference numbers will be used throughout the drawings to refer to the same or similar parts.

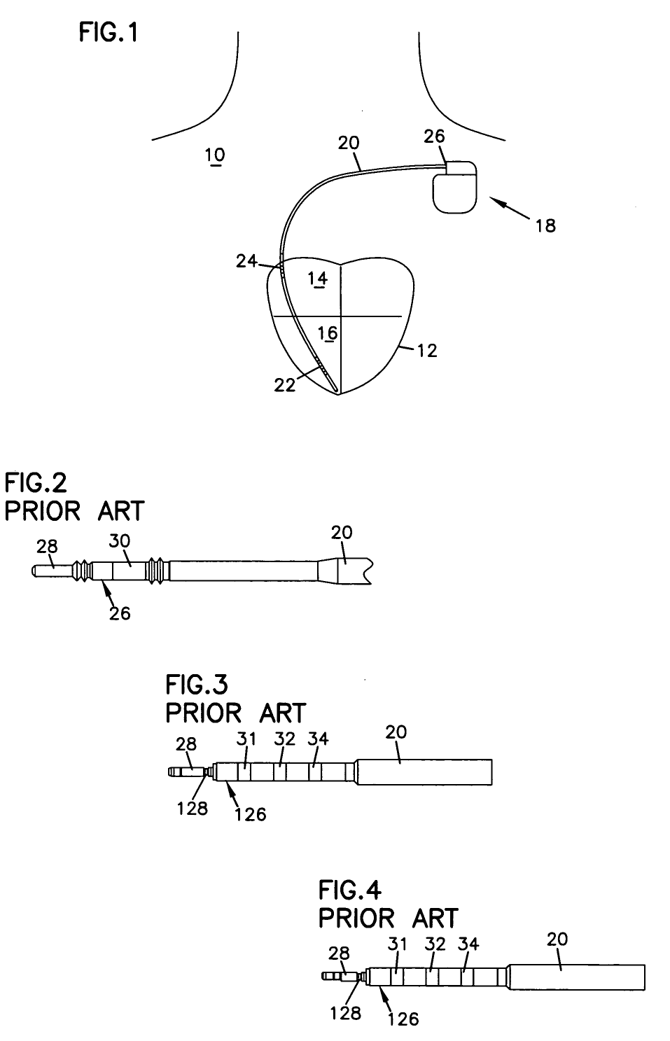

Implanted pulse generating devices, such as pacemaker 18 of FIG. 1, may be implanted in a body of a patient 10 to provide rhythm correcting electrical stimulus to the patient's heart 12. Such rhythm correcting techniques and devices are well known in the field of cardiac medicine. Pacemaker 18 is connected to heart 12 by a cardiac lead 20. Lead 20 includes a distal end with an electrode 22 inserted within or adjacent to one or more heart chambers, such as chambers 14 and 16. Lead 20 includes one or more electrodes 24 proximate distal end electrode 22 and corresponding conductors within lead 20 to carry electrical pulses from pacemaker 18 to heart 12.

Pacemaker 18 and lead 20 are physically implanted beneath the skin of patient 10. It is desirable that patient 10's pacemaker 18 not require frequent access for maintenance or repair, as such access may require that an invasive procedure be used. As such, testing of the connectivity and placement of lead 20 is preferred, prior to completion of an implantation procedure and closing any incision made during the procedure. Such testing will verify that one or more contacts, such as a pin contact 28, and ring contacts 31, 32 and 34, located at a terminal end 26 of lead 20 (shown in FIGS. 2 to 5) are in electrical contact with electrodes 22 and 24 and that electrodes 22 and 24 are properly positioned about the heart. Such testing may be performed with a Pacing System Analyzer (PSA), which is connected to terminal end 26 of lead 20. The PSA is connected by a PSA testing cable, or a patient cable, to one or more of the contacts of the terminal end and verifies the function of lead 20.

FIG. 2 illustrates terminal end 26 of lead 20 which conforms to an industry standard, known as IS-1. FIG. 2 shows a prior art dual contact (pin contact 28 and ring contact 30) terminal end 26. FIGS. 3 and 4 illustrate terminal ends 26 conforming to a potential forthcoming industry standard for quadripolar leads. Both FIGS. 4 and 5 show terminal ends 126 including a total of four contacts (pin contact 28 and three ring contacts 31, 32, and 34). FIG. 3 illustrates a configuration of terminal end 126 adapted for relatively lower voltage applications and FIG. 4 illustrates a configuration of terminal end 126 adapted for relatively higher voltage applications.

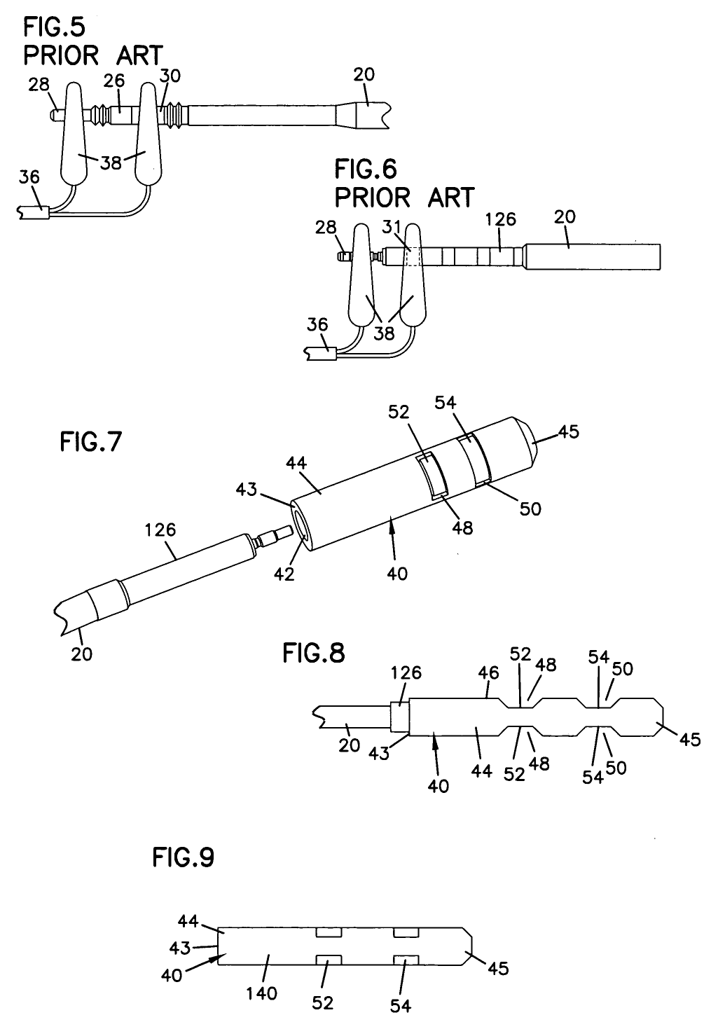

As shown in FIG. 5, a PSA cable 36 is connected to pin contact 28 and ring contact 30 of a dual contact terminal end 26 by a pair of spring clips, such as alligator clips 38. Such clips 38 are used to quickly and securely attach PSA cable 36 to lead 20 for testing and then to allow easy release so that terminal end 26 may be inserted within pacemaker 18, as shown in FIG. 1. FIG. 6 shows a quad contact terminal end 126 with PSA cable 36 connected to pin contact 28 and ring contact 31 by spring clips 38 for testing. The potential forthcoming industry standard for quadripolar leads requires that ring contact 31 be smaller in axial length than ring contact 30. While spring clips 38 may be correctly sized for connection to the IS-1 standard terminal end 26, the same clips 38 may be too large for optimal connection to a quadripolar terminal end 126. As IS-1 terminal ends 26 are in widespread use and will likely continue to be implanted in parallel with the newer quadripolar terminal ends 126, it is expected that these existing PSA cables 36 will still continue to be used with the current standard terminal ends 26. It is therefore desirable to have an adapter for use with quadripolar terminal ends 126 to permit use of existing PSA cables 36 with clips 38 for testing the quadripolar leads.

It should be noted that the adapters and cables described below are not intended for implantation within a patient's body but are for testing the leads following positioning of the distal end 22 adjacent or within the patient's heart. Once testing has been completed, the adapter and testing cables are removed from terminal end 126 and lead 20 is connected to pacemaker 18.

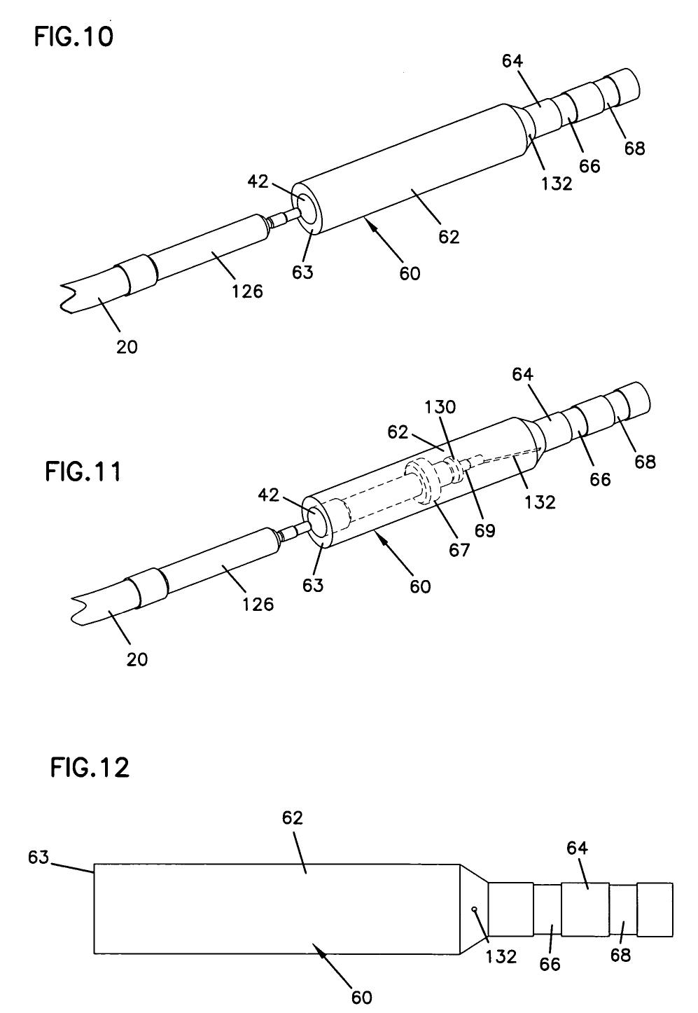

A first embodiment of such an adapter 40 is shown in FIGS. 7 to 9. Adapter 40 includes a central axial opening 42 defined by a housing 44, with opening 42 sized and configured to receive and mate with quadripolar terminal ends 126 through a first end 43. Opposite first end 43 is a second closed end 45, so that opening 42 is a blind opening which does not extend through adapter 40. While opening 42 does not extend entirely through adapter 40, a smaller opening 132 (shown in FIGS. 10 to 12 below) may be provided proximate second end 45 to permit air or other fluids within opening 42 to escape upon insertion of terminal end 126.

As shown in FIG. 8, housing 44 includes an outer surface 46 with a pair of recesses 48 and a pair of recesses 50. At the bottom of each recess 48 is a clip contact 52. Clip contact 52 is electrically connected to an inner contact within opening 42 which is electrically linked to ring contact 31 of terminal end 126 when terminal end 126 is fully inserted within opening 42. At the bottom of each recess 50 is a clip contact 54. Clip contact 54 is electrically connected to an inner contact within opening 42 which is electrically linked to pin contact 28 of terminal end 126 when terminal end 126 is fully inserted within opening 42. Recesses 48 and 50 are sized to receive spring clips 38 of PSA cable 36 and permit exiting cables 36 to be used for testing leads 20 including quadripolar terminal ends 126.

It is anticipated that outer surface 46 may be sized so that existing spring clips 38 may open wide enough to not require any recesses 48 or 50 and clip contacts 52 and 54 may be positioned directly on outer surface 46. An alternative configuration 140 of adapter 40 is shown in FIG. 9. Adapter 140 is similar to adapter 40 except that opposing pairs of clip contacts 52 and clip contacts 54 are located on outer surface 46. Use of adapter 140 would be permitted with spring clips 38 which can open wide enough for connecting to clip contacts 52 and 54 on outer surface 46.

Some PSA cables 36 may include spring clips 38 which are sized to fit very closely the diameter of IS-1 terminal end 26. Quadripolar terminal ends 126 are very similar in diameter to IS-1 terminal end 26. However, outer surfaces 46 of adapters 40 and 140 are by necessity larger in diameter than terminal end 126, as terminal end 126 is received within opening 42 of housing 44. Clip contacts 52 and 54 may be positioned within recesses 48 and 50, but the bottom of these recesses must still be displaced from each other a distance greater than the diameter of quadripolar terminal end 126 inserted within opening 42.

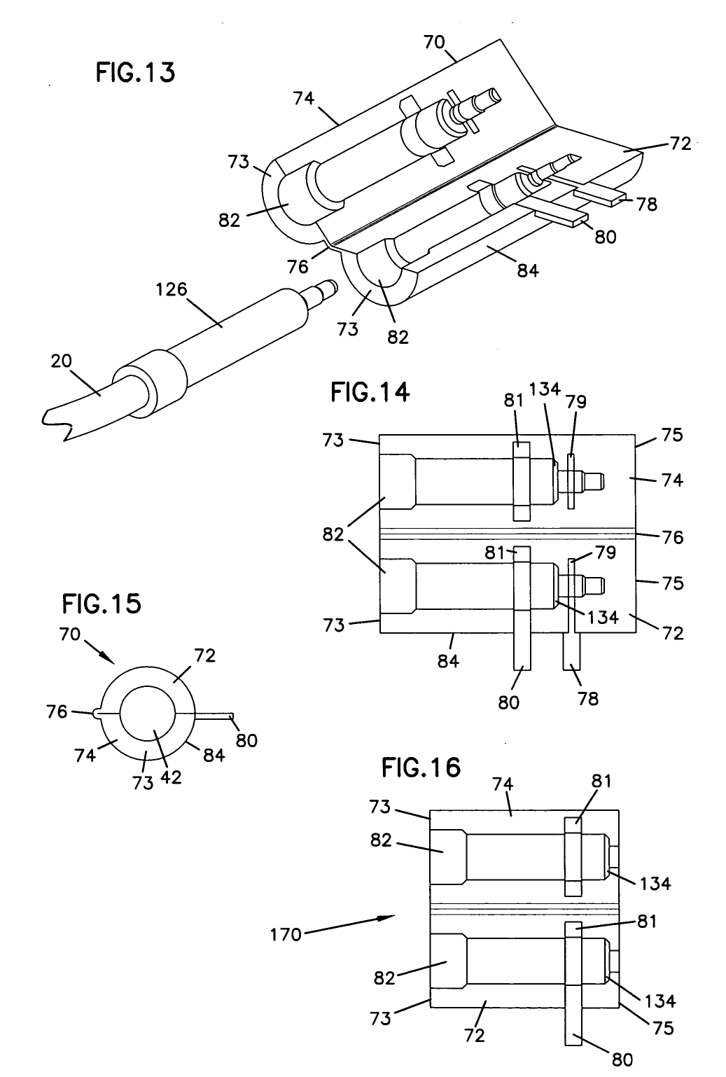

A second embodiment adapter 60 is shown in FIGS. 10 to 12, and includes central axial opening 42 within an housing 62 for receiving and mating with quadripolar terminal ends 126. Housing 62 also includes an extension 64 with a reduced diameter that approximates the diameter of terminal ends 26 and 126, opposite a first end 63. Extension 64 closes off the end of adapter 60 opposite first end 63 so that opening 42 does not extend through adapter 60. Quadripolar terminal end 126 is received within opening 42 through first end 63. Opening 132 allows air trapped within opening 42 by insertion of terminal end 126 to escape. Extension 64 includes a pair of clip contacts 66 and 68, sized for receiving spring clips 38 of PSA cable 36. Clip contact 66 is electrically connected to a first inner contact 67 within opening 42. First inner contact 67 is electrically linked to ring contact 31 of terminal end 126 when terminal end 126 is fully inserted within opening 42. Clip contact 68 is electrically connected to a second inner contact 69 within opening 42. Second inner contact 69 is electrically linked to pin contact 28 of terminal end 126 when terminal end 126 is fully inserted within opening 42. As shown in FIG. 11, a spring clip 130 is provided within opening 42. Spring 130 will be discussed further with regard to FIGS. 13 to 15.

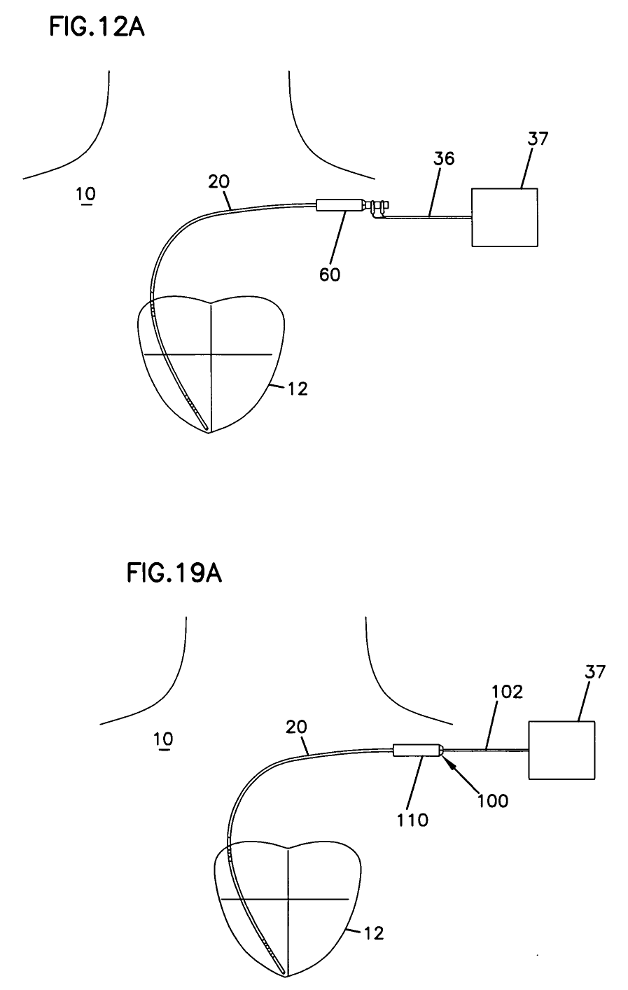

FIG. 12A shows lead 20 engaged by adapter 60 and positioned within heart 12. PSA cable 36 is connected to adapter 60 and to a PSA 37. As shown, PSA 37 can be used to test the function and positioning of lead 20.

A third embodiment 70 of an adapter for use with quadripolar terminal end 126 is shown in FIGS. 13 to 15. Adapter 70 includes a pair of adapter halves 72 and 74. Each half includes a recess 82 and the two halves are hingedly connected along one side by a hinge 76. In FIGS. 13 and 14, halves 72 and 74 are in an open position. As shown, hinge 76 is a living hinge but other types and styles of hinges may be used. In use, a terminal end 126 is placed within one of the recesses 82 and the two halves are brought together about hinge 76. As shown in FIG. 15, when halves 72 and 74 are in a closed position, the two recesses 82 cooperate to form central axial opening 42. Adapter 70 has a first end 73 from which terminal end 126 and lead 20 extend and an opposite second closed end 75, so that opening 42 does not extend through adapter 70. No opening 132 is required, as closing adapter halves 72 and 74 should not trap air within opening 42. However, adapter 70 may include opening 132 to aid insertion of terminal end 126 when adapter 70 is closed, as shown in FIG. 15.

Each of the halves includes a first inner contact 79 which is electrically linked with pin contact 28 of terminal end 126 when terminal end 126 is positioned within opening 42. When halves 72 and 74 are closed about terminal end 126, the two first inner contacts 79 are in electrical contact with each other and with a first clip tab 78 extending from an adapter outer surface 84. First clip tab 78 is sized and configured to be engaged with one of the spring clips 38 of PSA cable 36.

Each of the halves also includes a second inner contact 81 which is electrically linked with ring contact 31 of terminal end 126 when terminal end 126 is positioned within opening 42. When halves 72 and 74 are closed about terminal end 126, the two second inner contacts 81 are in electrical contact with each other and with a second clip tab 80 extending from an adapter outer surface 84. Second clip tab 80 is sized and configured to be engaged with one of the spring clips 38 of PSA cable 36.

Alternatively, the inner contacts of adapter 70 may be electrically linked to outer contacts such as included with adapter 40, described above. Instead of providing tabs 79 and 81 extending from the side of adapter 70, adapter 70 could include outer contacts 52 and 54 on outer surface 84. Outer contacts 52 and 54 could also be mounted within recesses similar to recesses 48 and 50 of adapter 40.

As a further alternative, outer contacts 78 and 80 may be rounded extensions, or may be formed in some other shape than the flat tab extensions shown in the FIGS.

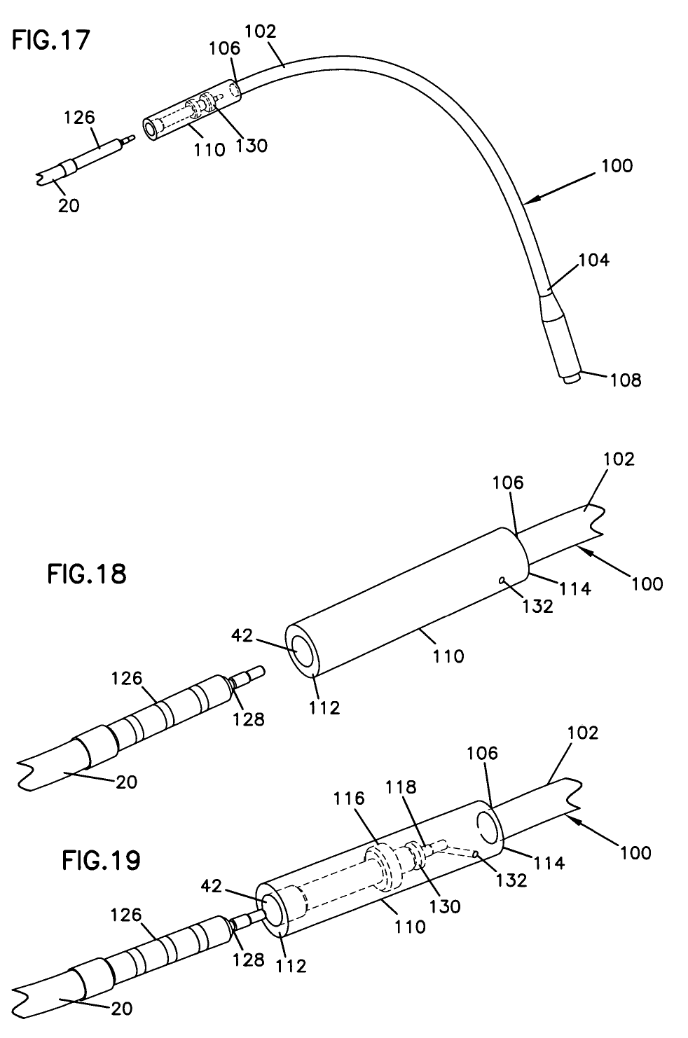

Within each recess 82 is a spring clip area 134. Terminal end 126 includes a recess 128 between tip contact 28 and ring contact 31 (as shown in FIGS. 3 and 4, above). During testing of lead 20 with the PSA, it is desirable that the connection between lead 20 and PSA cable 36 be as secure as possible. A mechanical retention mechanism, such as a portion of a spring clip 130 (not shown in FIGS. 13 to 16, shown in FIG. 11, above, and in FIGS. 17 and 19, below) may be positioned in spring clip area 134 of each recess 82 and may releasably engage recess 128 when halves 72 and 74 are closed about terminal end 126. Spring clip 130 will ensure that adapter 70 remains securely mounted to terminal end 126. As shown in FIG. 11, and FIGS. 17 and 19, spring clip 130 may also be positioned within opening 42 of other embodiments of adapters according to the present invention.

It is anticipated that other mechanical retention mechanisms or devices may be provided within adapters according to the present invention. These other mechanisms or devices may include but are limited to a set screw extending through housing 44 to engage terminal end 126, or o-rings or other deformable materials within axial opening 42 which conform to a portion of terminal end 126 and releasably hold the adapter to terminal end 126. Such retention mechanisms or devices may be positioned between contacts of terminal end 126, provided they do not interfere with electrical contact of pin contact 28 or ring contact 31. Alternatively, such retention mechanisms or devices may be positioned proximal to ring contact 134. Such mechanisms or devices may be cooperating shapes or features both within the adapter and on terminal end 126, (such as recess 128 and spring clip 130) or may be features or shapes included solely on one or the other.

FIG. 16 shows an alternative configuration of adapter 170, with opening 42 extending through second end 75. Instead of having first clip tab 78 extending from outer surface 84 and first inner contact 79 within opening 42 or recesses 82, pin contact 28 is positioned to extend beyond second end 75. So positioned, clip 38 of PSA cable 36 may be placed directly about pin contact 28. Spring clip 130 may not be required to secure hold adapter 170 to terminal end 126, as the clip 38 positioned about tip contact 28 might rest against second end 75 and reduce movement of terminal end 126 within opening 42. Clip 130 may be provided within recesses 82 and opening 42 to provide greater security against undesirable movement.

As discussed above, it may be desirable to have an adapter which permits existing PSA testing cables to be connected to different styles and standards of cardiac lead terminal ends. However, not all testing cables and testing situations require such multiple terminal end compatibility. FIGS. 17 to 19 show a cable assembly 100 adapted for use directly with terminal end 126 of cardiac lead 20. Cable assembly 100 includes a cable 102 with a proximal end 104 and a distal end 106. Mounted at proximal end 104 is a connector end 108 for connection to a testing device such as a PSA. Mounted to distal end 106 is a terminal end receptacle or adapter 110. Terminal end 126 is inserted into opening 42 through a first end 112. Terminal end adapter 110 is connected to distal end 106 of cable 102 at a second closed end 114.

Adapter 110 includes a central axial opening 42 configured like the commonly numbered openings 42 of the adapters above. However, instead of providing for external connection of spring clips 38 of a PSA cable 36, the inner contacts of opening 42 are connected to conductors within cable 102 and through connector end 108 to a testing device. As shown in FIG. 19, a first inner contact 116 of opening 42 is electrically linked to ring contact 31 of terminal end 126 and a second inner contact 118 is electrically linked to pin contact 28 of terminal end 126, when terminal end 126 is within opening 42. FIG. 19A shows cable assembly 100 connected to lead 20 and to PSA 37. Lead 20 has been positioned within the patient's heart and PSA 37 can be used to test the positioning and function of lead 20.

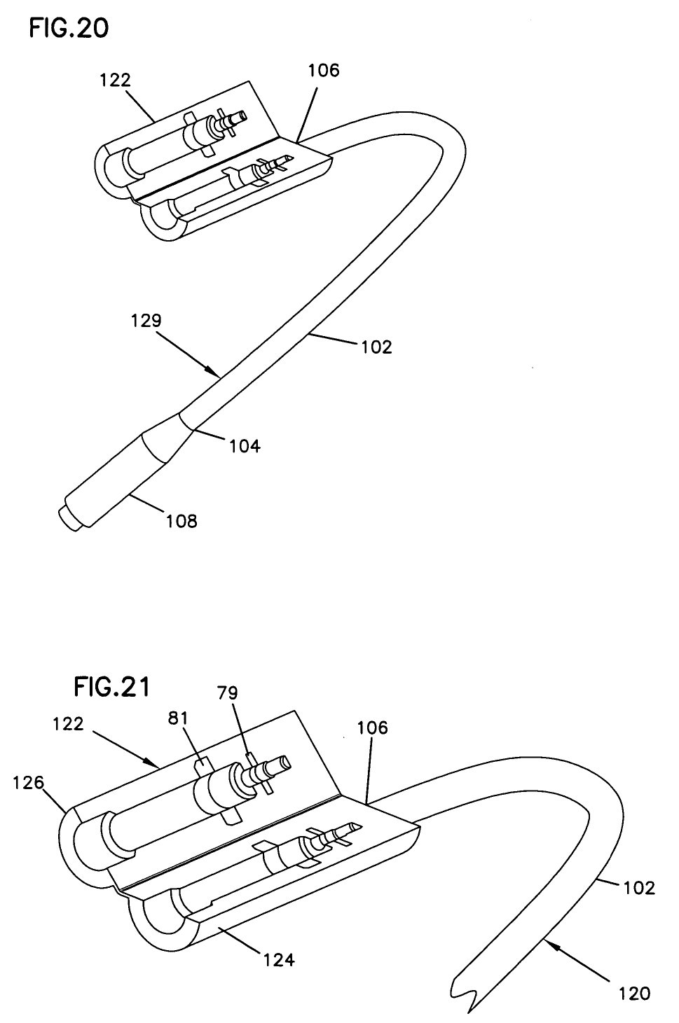

FIGS. 20 and 21 show a second embodiment 120 of a cable assembly with a terminal end adapter 122 mounted to distal end 106 of cable 102. Terminal end adapter 122 is similar to adapter 70, described above, with the exception of the external clip tabs. In place of the external clip tabs of adapter 70, terminal end adapter 122 electrically links first inner contacts 79 and second inner contacts 81 with conductors within cable 102 and through connector end 108 to a testing device. Otherwise, the features and construction of a first half 124 and a second half 126 are similar to halves 72 and 74, respectively of adapter 70.

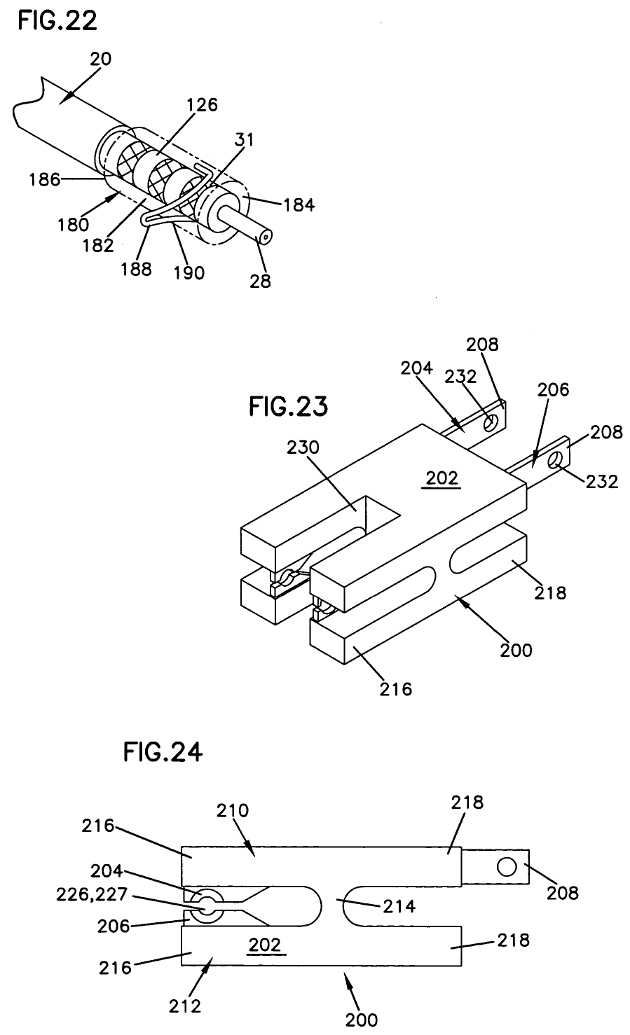

FIG. 22 shows a further embodiment of an adapter 180 for attaching clips 38 of PSA cable 36 to terminal end 126 of lead 20. Adapter 180 includes a sleeve 182 positioned about terminal end 126. Pin contact 28 extends beyond a second end 184 while a portion of terminal end 126 is within opening 42, and the remainder of lead 20 extends beyond a first end 186. One of the clips 38 of PSA cable 36 can be connected directly to pin contact 28. The other clip 38 is electrically connected to ring contact 31 by a conductor 188 extending from opening 42 beyond an outer surface 190 of sleeve 182.

FIGS. 23 to 26 show a lead clip 200 for electrically connecting spring clips 38 of PSA cable 36 to pin contact 28 and ring contact 31 of terminal end 126 of lead 20. Lead clip 200 includes a body 202 of a dielectric material and a pair of clip contacts 204 and 206 mounted to body 202 for electrically connecting to pin contact 28 and ring contact 31, respectively. Each clip contact 204 and 206 includes a spring clip end 208 to which spring clips 38 are connected. Clip ends 208 may include a feature such as an opening 232 to improve the physical hold of spring clip 38 to the contacts. Other features may be used on clip ends 208 to provide similar enhanced grip, including but not limited to dimples, serrations, protrusions, or checkering.

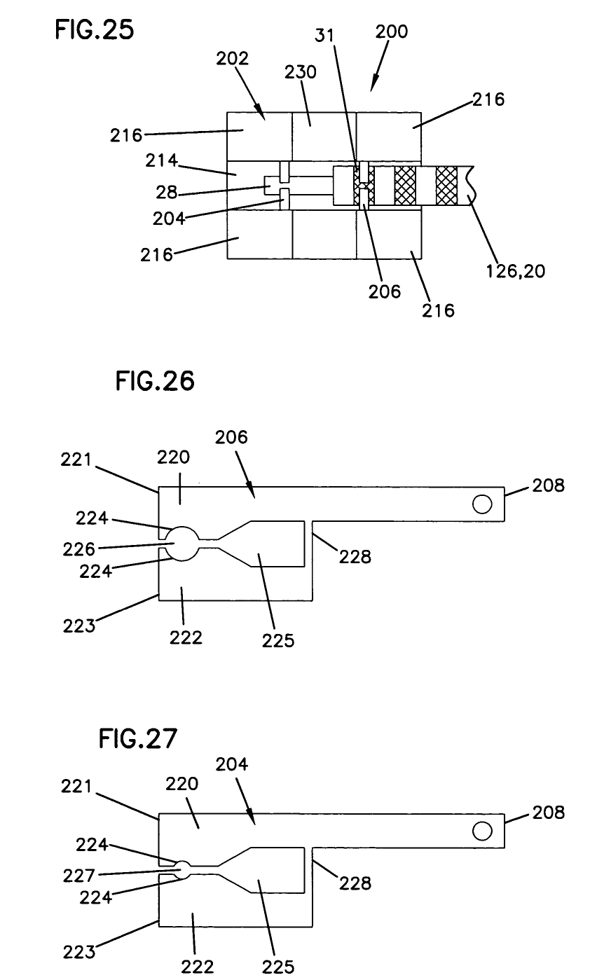

Lead clip 200 includes an upper portion 210 and a lower portion 212, separately and connected by a web 214. Web 214 is sufficiently flexible to permit a physician or other user of the clip to apply compressive force on a rear end 218 to force front end 216 to open and receive terminal end 126. Once terminal end 126 has been positioned with pin contact 28 adjacent clip contact 204 and ring contact 31 adjacent clip contact 206, pressure on rear end 218 can be released and lead clip 200 engages terminal end 126, as shown in FIG. 25.

Clip contact 206 is shown in FIG. 26, removed from lead clip 200, and includes an upper leg 220, extending from a first end 221 to clip end 208, and a lower leg 222, extending from a first end 223 to a contact web 228. Contact web 228 extends between upper leg 220 and lower leg 222 and is within web 214 when contact 206 is assembled as part of lead clip 200. Web 228 is flexible enough to permit front end 216 to open when compressive force is applied to rear end 218 of lead clip 200. Between contact web 228 and first ends 221 and 223 is defined an opening 225 between upper leg 220 and lower leg 222. Adjacent first ends 221 and 223 within opening 225 are a pair of half round openings 224, cooperating to form a terminal opening 226. Terminal opening 226 is sized to fit about and contact ring contact 31 of terminal end 126.

Shown in FIG. 27, clip contact 204 is similarly configured, defining a terminal opening 227 sized to fit about and contact pin contact 28 of terminal end 126.

As shown in the FIGS., body 202 of clip 200 includes a notch or recess 230 in the upper and lower legs 210 and 212, positioned between clip contacts 204 and 206. While there may be advantages to having recess 230, such as improving visibility for positioning clip 200 about terminal end 126, recess 230 is not required.

Body 202 can be formed of any dielectric material of sufficient strength to permit the application of compressive force on rear ends 218 to open clip 200 for placement about terminal end 126, but flexible enough to permit web 214 to remain intact during opening and closing of clip 200. Alternatively, clip body 202 could be composed of upper leg 210, lower leg 212 and web 214 as separate components, each made of materials having suitable physical and electrical qualities for clip 200 to operate as described above.

While the discussion above has described terminal ends of cardiac leads as conforming to one of several existing standards, it is anticipated that new and different terminal ends will be and have been developed. The adapters and cable assemblies of the present disclosure may be adapted for use with these new and/or different terminal configurations without straying from the bounds of the present invention.

Having described preferred aspects and embodiments of the present invention, modifications and equivalents of the disclosed concepts may readily occur to one skilled in the art. However, it is intended that such modifications and equivalents be included within the scope of the claims which are appended hereto.

1. An adapter for an implantable cardiac lead comprising:
a housing with an interior axial opening and an outer surface, the interior axial opening configured to receive a mating terminal end of the implantable cardiac lead;
a plurality of inner contacts within the interior axial opening and a plurality of outer contacts on the outer surface, each of the inner contacts electrically connected to an outer contact;
the inner contacts configured to electrically link with contacts on the mating terminal end of the implantable cardiac lead when the mating terminal end of the implantable cardiac lead is positioned within the interior opening;
the axial opening including a mechanical retention means to releasably hold the housing to the terminal end; and
the outer surface configured for physical and electrical connection of the outer contacts to a testing cable in communication with a device for testing electrical aspects of the cardiac lead.

2. The adapter of claim 1, wherein the adapter is releasably mounted to the testing cable for testing the electrical continuity of the cardiac lead.

3. The adapter of claim 2, wherein each of the outer contacts of the adapter are connected to the testing cable by an alligator clip on the cable.

4. The adapter of claim 1, wherein the terminal end of the implantable cardiac lead includes four contacts, the housing includes two inner contacts and two outer contacts, and insertion of the terminal end of the implantable cardiac lead within the interior opening of the housing electrically links two of the contacts of the terminal end with the two inner contacts and the two outer contacts of the adapter.

5. The adapter of claim 1, wherein the terminal end of the implantable cardiac lead includes two contacts, the housing includes two inner contacts and two outer contacts, and insertion of the terminal end of the implantable cardiac lead within the interior opening of the housing electrically links the two contacts of the terminal end with the two inner contacts and the two outer contacts of the adapter.

6. The adapter of claim 1, wherein the housing includes a first end through which the terminal end of the implantable cardiac lead is inserted within the interior opening and a closed second end, and further includes an opening adjacent the second end to permit air to escape the axial opening as the terminal end is inserted within the axial opening.

7. The adapter of claim 6, wherein outer surface of the housing includes a plurality of recesses with the outer contacts each positioned at a bottom of one of the recesses.

8. The adapter of claim 7, wherein the testing cable is connected to the outer contacts in the recesses of the outer surface by spring clips.

9. The adapter of claim 7, wherein a pair of outer contacts is electrically connected to each inner contact and each pair of outer contacts are positioned opposing each other on the outer surface of the housing within opposing recesses.

10. The adapter of claim 6, wherein the outer housing about the interior axial opening defines a first portion, the closed second end includes an axial extension which is smaller in diameter than the first portion, and the outer contacts are positioned on the axial extension.

11. The adapter of claim 10, wherein the axial extension is approximately the same diameter as the terminal end of the implanted cardiac lead.

12. The adapter of claim 6, wherein the housing includes a first half and second half, the halves hingedly connected to one another by an axially arranged hinge adjacent the outer surface of the housing, the halves movable between an open position and a closed position, and each half includes a interior recess, the interior recesses cooperating to form the interior axial opening when the halves are in the closed position.

13. The adapter of claim 12, wherein the outer contacts are tabs extending from the outer surface of the housing.

14. The adapter of claim 13, wherein the inner contacts are positioned within the interior recess of the first half of the housing.

15. The adapter of claim 14, wherein a second set of inner contacts are positioned within the recess of the second half of the housing and each inner contact in the recess of the first half is paired with one of the second inner contacts in the recess of the second half and each of the pairs of inner contacts and second inner contacts is electrically connected to one of the tabs of the outer contacts.

16. An assembly for testing an implanted cardiac lead comprising:
the cardiac lead including a distal end in electrical communication with a patient's heart and a proximal end including a terminal end adapted for connection to a pulse generating device, the terminal end including a plurality of contacts;
a testing cable adapted to communicate with a device for testing the electrical continuity of the cardiac lead, the testing cable having a distal end including a plurality of spring clips for linking to the terminal end of the implanted cardiac lead and a proximal end for connection to the testing device;
an adapter configured to fit about the terminal end of the implanted cardiac lead and provide electrical connection between the plurality of contacts of the terminal end and the plurality of spring clips mounted at the distal end of the testing cable; and,
the adapter positioned about the terminal end of the cardiac lead and providing an electrical connection between the spring clips of the testing cable attached to a plurality of outer contacts on the adapter and a plurality of contacts on the terminal end of the cardiac lead within the opening.

17. The assembly of claim 16, wherein the number of contacts of the terminal end of the implanted cardiac lead is greater than the number of spring clips of the testing cable.

18. The assembly of claim 16, wherein the adapter includes a housing defining an axial opening with a first end through which the terminal end of the implanted cardiac lead is received and a second closed end with an opening adjacent the second closed end to permit air to escape from the axial opening when the terminal end is inserted within the axial opening, the axial opening further includes a plurality of inner contacts for electrically linking to contacts of the terminal end, and the housing further includes an outer surface on which a plurality of outer contacts are positioned, each the inner contacts electrically connected to one of the outer contacts and the spring clips of the testing cable connected to the outer contacts.

19. The assembly of claim 18, wherein the outer contacts are positioned within recesses in the outer surface of the housing.

20. The assembly of claim 18, wherein the outer contacts include tabs extending from the outer surface of the housing.

21. The assembly of claim 18, wherein the housing includes a first portion about the axial opening and an extended portion adjacent the closed end which is smaller in diameter than the first portion, and the outer contacts are positioned on the outer surface of the housing on the extended portion.

22. The assembly of claim 18, wherein the housing of the adapter includes a pair of halves, the halves hingedly mounted to each other by a hinge and movable between an open position and a closed position, each half including a recess and the recesses cooperating to form the axial opening when in the closed position.

23. A cable assembly for connecting an implantable cardiac lead to a testing device, the cable assembly comprising:
an adapter for an implantable cardiac lead including a housing with an interior axial opening and an outer surface, the interior axial opening configured to receive a mating terminal end of the implantable cardiac lead, and a plurality of inner contacts within the interior axial opening, each inner contact configured to electrically link with one of a plurality of a contacts of the mating terminal end of the implantable cardiac lead when the mating terminal end of the implantable cardiac lead is positioned within the interior opening, the axial opening including a mechanical retention means to releasably hold the housing to the terminal end; and
a cable including a distal end to which the adapter is mounted, a proximal end for engaging the testing device, and a plurality of electrical conductors extending from the distal end to the proximal end, each of the inner contacts of the adapter electrically connecting one of the contacts of the mating terminal end with one of the conductors of the cable when the mating terminal end is inserted within the axial opening.

24. The cable assembly of claim 23, wherein the housing includes a first half and second half, the halves hingedly connected to one another by an axially arranged hinge adjacent the outer surface of the housing, the halves movable between an open position and a closed position, and each half includes a interior recess, the interior recesses cooperating to form the interior axial opening when the halves are in the closed position.

25. A method of connecting an implanted cardiac lead to a testing device comprising:
providing the implanted cardiac lead with a distal end positioned within a body of a patient and including a terminal end including a plurality of contacts;
providing a testing cable with a proximal end adapted for connection with the testing device and a distal end including a plurality of spring clips;
providing an adapter including a housing defining an axial opening with a first end and a closed second end, a plurality of inner contacts within the opening and a plurality of outer contacts electrically connected with the inner contacts on an outer surface of the housing;
placing the terminal end of the cardiac lead within the axial opening of the adapter so that contacts of the terminal end are electrically linked with the inner contacts; and,
connecting the spring clips of the distal end of the testing cable with the outer contacts of the adapter, so that the contacts of the terminal end are electrically connected to the spring clips of the testing cable.

26. A clip for attaching to the terminal end of a multiple conductor cardiac lead, the clip comprising:
a body having a front end and a rear end, and including an upper leg extending from the front end to the rear end, a lower leg extending from the front end to the rear end, the upper and lower legs spaced apart and a web extending between the upper and lower legs intermediate from the front and rear ends;
a plurality of clip contacts extending from adjacent the front end to the beyond the rear end, each contact defining a tab adjacent the rear end;
a first clip contact defining an opening between the upper and lower legs adjacent the front end configured to engage and electrically connect a pin contact of the terminal end of the cardiac lead with the tab of the first clip contact;
a second contact defining an opening between the upper and lower leads adjacent the front end configured to engage and electrically connect a ring contact of the terminal end of the cardiac lead with the tab of the second clip contact.
